(19) United States
(12) Patent Application Publication
(10) Pub. No.: US 2025/0241842 A1
(43) Pub. Date: Jul. 31, 2025

(54) THERMO-SENSITIVE COMPOSITION AND METHOD FOR PREPARATION THEREOF

(71) Applicant: BASF SE, Ludwigshafen (DE)

(72) Inventors: Zhi Hui Zhu, Shanghai (CN); Lan Liao, Shanghai (CN)

(21) Appl. No.: 18/688,889

(22) PCT Filed: Aug. 31, 2022
(86) PCT No.: PCT/EP2022/074206

§ 371 Date: Mar. 4, 2024

(30) Foreign Application Priority Data
Sep. 8, 2021 (WO) PCT/CN2021/117193

(51) Int. Cl. A61K 8/90; A61K 8/06; A61K 8/31; A61K 8/34; A61K 8/36; A61Q 19/00

(52) U.S. Cl. CPC A61K 8/90; A61K 8/06; A61K 8/31; A61K 8/345; A61K 8/361; A61Q 19/00; A61K 2800/10; A61K 2800/24

(57) ABSTRACT

The present invention relates to a thermo-sensitive composition comprising i) a water-soluble block copolymer comprising at least two blocks of polyethylene oxide and at least one block of polypropylene oxide; and (ii) at least one ester-based emollient and/or alkane-based emollient, especially for the delivery of cosmetics active ingredients, and method for preparation thereof.

### FIELD OF THE INVENTION

The present invention relates to a thermo-sensitive composition, especially for the delivery of cosmetics active ingredients, and method for preparation thereof.

### BACKGROUND

Thermo-sensitive systems in cosmetics application can deliver many unique benefits, for example texture transform, smart viscosity control, active release control, heat force SPF boost, film formation and homogenous powder distribution.

Water-soluble block copolymers of polyethylene oxide and polypropylene oxide are classical series of materials which have thermo-sensitive behavior.

Currently the main application of these materials which have thermo-sensitive behavior is solubilization and active release control in personal care and pharmaceutical area. Some prior arts are also focused on hydrogel compositions containing water-soluble block copolymers of polyethylene oxide and polypropylene oxide, which are in a liquid state at room temperature or below, subsequently transform to a gel form when warmed to body temperature after application.

U.S. Pat. No. 4,188,373A discloses the use of non-ionic block copolymers of polyethylene oxide and polypropylene oxide in aqueous pharmaceutical compositions. In these systems, the concentration of polymer is adjusted to give the desired sol-gel transition temperature.

U.S. Pat. No. 8,865,143B discloses a reversely thermo-reversible hydrogel composition, having a gel form in the temperature range of from about 4 to 50° C.

However, the thermo-sensitive application of the water-soluble block copolymers of polyethylene oxide and polypropylene oxide in emulsion system is still rare on the market. In the gel type formats, normally high level of the water-soluble block copolymers of polyethylene oxide and polypropylene oxide is required to deliver the thermo-sensitive behavior which leads to unpleasant sensory, higher cost and less formula compatibility.

### SUMMARY OF THE INVENTION

It is an object of the present invention to provide an optimized formulation system by selecting specific emollients to get the expected thermo-sensitive behavior with less content of water-soluble block copolymers of polyethylene oxide and polypropylene oxide in the composition.

It has been found that the object of the present invention can be achieved by a thermo-sensitive composition comprising (a) a water-soluble block copolymer comprising at least two blocks of polyethylene oxide and at least one block of polypropylene oxide; (b) at least one ester-based emollient and/or alkane-based emollient; and (c) at least one emulsifier, wherein said composition has a texture transition temperature from 5 to 40° C.

Particularly, the present invention relates to following aspects.

In a first aspect, the present invention provides a thermo-sensitive composition comprising (a) a water-soluble block copolymer comprising at least two blocks of polyethylene oxide and at least one block of polypropylene oxide; (b) at least one ester-based emollient and/or alkane-based emollient; and (c) at least one emulsifier, wherein said composition has a texture transition temperature from 5 to 40° C.

In a second aspect, the present invention provides a method for preparing the thermo-sensitive composition according to the present invention, comprising mixing the components of the thermo-sensitive composition.

In a third aspect, the present invention provides the use of the thermo-sensitive composition according to the invention as a delivery system for cosmetically active ingredients.

### DETAILED DESCRIPTION OF THE INVENTION

The present invention now will be described in details hereinafter. It is to be understood that the present invention may be embodied in many different ways and shall not be construed as limited to the embodiments set forth herein. Unless mentioned otherwise, all technical and scientific terms used herein have the same meaning as commonly understood by one of ordinary skill in the art to which this invention belongs.

As used herein, the singular forms “a”, “an”, and “the” include plural referents unless the context clearly dictates otherwise.

As used herein, the terms “comprise”, “comprising”, etc. are used interchangeably with “contain”, “containing”, etc. and are to be interpreted in a non-limiting, open manner. That is, e.g., further components or elements may be present. The expressions “consists of” or “consists essentially of” or cognates may be embraced within “comprises” or cognates.

The first aspect of the present invention provides a thermo-sensitive composition comprising (a) a water-soluble block copolymer comprising at least two blocks of polyethylene oxide and at least one block of polypropylene oxide; (b) at least one ester-based emollient and/or alkane-based emollient; and (c) at least one emulsifier, wherein said composition has a texture transition temperature from 5 to 40° C. Particularly, for example, the texture transition is from liquid texture to semi-solid or solid texture. More particularly, for example, the texture transition is from lotion or flowing emulsion texture to cream texture.

Preferably, the thermo-sensitive composition according to the present invention has a texture transition (for example from lotion to cream) temperature from 6 to 40° C., for example 10° C., 15° C., 20° C., 25° C., 30° C., 35° C., more preferably from 15 to 40° C.

Water-Soluble Block Copolymer (a) In the context of the present invention, the thermo-sensitive composition comprises a water-soluble block copolymer comprising at least two blocks of polyethylene oxide and at least one block of polypropylene oxide as component (a).

The terms “polyethylene oxide”, “PEO”, “EO”, “polyethylene glycol”, and “PEG” are used interchangeably to describe the present invention, and refer to synthetic polymer of ethylene oxide represented by the following chemical structure:

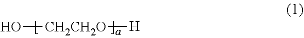

in which a is an integer representing the average number of monomer repeating units.

The terms “polypropylene oxide”, “PPO”, “PO”, “polypropylene glycol”, and “PPG” are used interchangeably to describe the present invention, and refer to synthetic polymer of propylene oxide represented by the following chemical structure:

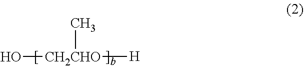

in which b is an integer representing the average number of monomer repeating units.

Block copolymer of polyethylene oxide and polypropylene oxide refers to a synthetic copolymer of polyethylene oxide block (Formula 1) and polypropylene oxide block (Formula 2), of varying molecular weights, and of various types, ranging from linear multi-block copolymers, side-chain grafted block copolymers, and hyper-branched block copolymers to star-shaped block copolymers; The block copolymers of polyethylene oxide and polypropylene oxide also comprise end-modified and chain-extended block copolymers of various types.

Of particular interest water-soluble block copolymers of present invention are the block copolymers comprising at least two blocks of polyethylene oxide of the formula —[CH₂CH₂O]ₐ—, and at least one block of polypropylene oxide of the formula —[CH₂CH(CH₃)O]_{b}—, where a represents a number from about 10 to 160, preferably from about 40 to 160, more preferably from about 50 to 150, most preferably from about 55 to 145, and b represents a number from about 10 to 160, preferably from about 30 to 80, more preferably from about 35 to 70, most preferably from about 40 to 60.

Exemplary water-soluble block copolymers comprising at least two blocks of polyethylene oxide and at least at least one block of polypropylene oxide of present invention are tri-block copolymers commercially available under the trade name PLURONIC®, also known as Poloxamer from BASF Corporation, Mount Olive, N.J. A preferred Poloxamer polymers, having the general formula of HO-(EO)ₐ(PO)_{b}(EO)ₐ—H, are PLURONIC® F127 (also known as Poloxamer 407) with average values of a at about 101, and b at about 56, and PLURONIC® F108 (also known as Poloxamer 338) with average values of a at about 141, and b at about 44, respectively.

Other exemplary water-soluble block copolymers comprising at least two blocks of polyethylene oxide and at least one block of polypropylene oxide of present invention are the linear multi-block copolymer, having the general formula of HO—[(PO)_{b}(EO)ₐ]m(PO)_{c}[(EO)ₐ(PO)_{b}]ₘ—H, where (EO)ₐ is a polyethylene oxide block, and (PO)_{b} or (PO)_{c} is a polypropylene oxide block, a, b and c each represent a number from about 10-160, and m is the integer value greater than 0.

Other water-soluble multi-block copolymers of present invention are chain extended, hyper-branded, or star-shaped block copolymers of the formula {[Aₙ(EO)ₐ(PO)_{b}(EO)ₐAₙ]E}ₘ, where (EO)ₐ is a polyethylene oxide block, and (PO)_{b} is a polypropylene oxide block, A is a monomer repeating unit, E is a chain extender or crosslinking agent, n is an integer ranging from 0 to 50, preferably 1 to 20 (0 to 20 in the case of non-biodegradable materials), even more preferably 2 to 16 (0 to 16 in the case of non-biodegradable materials), and m is the number of repeating units in the polymer molecule and is an integer equal to or greater than 2 (within practical limits, up to about 100,000 or more), preferably ranging from about 2 to about 500, more preferably about 3 to 100. Thus, where n is 0, the present invention contemplates polymers of the structure {[(EO)ₐ(PO)_{b}(EO)ₐ]E}m.

Other water-soluble block copolymer of present invention is an end-modified block copolymers of general formula R-G-(EO)ₐ(PO)_{b}(EO)ₐ-G-R, where (EO)ₐ is a polyethylene oxide block, and (PO)_{b} is a polypropylene oxide block, G is selected from a group consisting of C—C, C—O, C(O)NH, S—C, C(O)—O, and Si—O, R is alkyl or arylalkyl with alkyl chain length in the range of C₆-C₃₆, a is an integer ranging from 50 to 150, b is an integer ranging from 35 to 70.

Exemplary water-soluble block copolymers are the alkyl or arylalkyl end-modifiers block copolymers comprising at least two blocks of polyethylene oxide and at least one block of polypropylene oxide, which are a product of alcohol condensation reactions with a terminal alkyl or arylalkyl group. The alkyl group should have hydrophobic character, such as butyl, hexyl and the like. An alkyl poloxamer may have the general formula R-[(EO)ₐ(PO)_{b}(EO)ₐ]ₘ-R′, where (EO)ₐ is a polyethylene oxide block, (PO)_{b} is a polypropylene oxide block, R and R′ are the nonpolar groups, such as alkyl and arylalkyl with alkyl chain length in the range of C₆-C₃₆, and m is an integer ranging from 1 to 10.

Other exemplary water-soluble block copolymers of present invention are the grafted block copolymers comprising grafted side chain of at least two blocks of polyethylene oxide and at least one block of polypropylene oxide, having general formula of [A(EO)ₐ(PO)_{b}(EO)ₐ]ₘ, where (EO)ₐ is a polyethylene oxide block, (PO)_{b} is a polypropylene oxide block, a and b each independently represents a number from about 10 to 150. A is a monomer repeating unit of backbone of the copolymers consisting of vinyl, ester, amide, imide, ether, siloxane linkages, and the like, m is the number of repeating units in the polymer molecule and is an integer equal to or greater than 2.

Other water-soluble multi-block copolymers of present invention are polyester chain extended block copolymers of the formula {[Aₙ(EO)ₐ(PO)_{b}(EO)ₐAₙ]E}ₘ, where (EO)ₐ is a polyethylene oxide block, (PO)_{b} is a polypropylene oxide block, A is a monomer repeating unit, (EO)ₐ is a polyethylene oxide block, and (PO)_{b} is a polypropylene oxide block as previously defined, E is a chain extender or crosslinking agent, n is an integer ranging from 0 to 50, preferably 1 to 20 (0 to 20 in the case of non-biodegradable materials), even more preferably 2 to 16 (0 to 16 in the case of non-biodegradable materials) and m is the number of repeating units in the polymer molecule and is an integer equal to or greater than 2 (within practical limits, up to about 100,000 or more), preferably ranging from about 2 to about 500, more preferably about 3 to 100. Thus, where n is 0, the present invention contemplates polymers of the structure {[(EO)ₐ(PO)_{b}(EO)ₐ]E}m.

The monomer repeating units may be derived from an aliphatic hydroxy carboxylic acid or a related ester, lactone, dimeric ester, carbonate, anhydride, dioxanone, amide, or related monomer, and preferably derived from an aliphatic α-hydroxy carboxylic acid or related ester, such units derived from the following: including, for example, lactic acid, lactide, glycolic acid, glycolide, or a related aliphatic hydroxyl carboxylic acid, ester (lactone), dimeric acid or related compound such as, for example, β-propiolactone, ε-caprolactone, δ-glutarolactone, δ-valerolactone, β-butyrolactone, pivalolactone, α,α-diethylpropiolactone, ethylene carbonate, trimethylene carbonate, γ-butyrolactone, p-dioxanone, 1,4-dioxepan-2-one, 3-methyl-1,4-dioxane-2,5-dione, 3,3,-dimethyl-1-4-dioxane-2,5-dione, cyclic esters of α-hydroxybutyric acid, α-hydroxyvaleric acid, α-hydroxyisovaleric acid, α-hydroxycaproic acid, α-hydroxy-α-ethylbutyric acid, α-hydroxyisocaproic acid, α-hydroxy-α-methyl valeric acid, α-hydroxyheptanoic acid, α-hydroxystearic acid, α-hydroxylignoceric acid, salicylic acid and mixtures thereof. The use of α-hydroxyacids and their corresponding cylic dimeric esters, especially lactide, glycolide, and caprolactone in the present invention, is preferred. It is noted that in using certain of the described monomers according to the present invention, the monomeric units which are produced are not specifically ester groups, but may include such groups as carbonate groups (polycarbonates), amino acids (which produce polyamides) and related groups which are derived from the above-described monomers or which contain a nucleophilic group and an electrophilic group and can be polymerized. It will be understood that the term polyester shall encompass polymers which are derived from all of the above monomers, with those which actually produce ester units being preferred.

The terms “poly(hydroxy carboxylic acid)” or “poly(α-hydroxy carboxylic acid)” are terms used to describe certain polyester A blocks of the {[Aₙ(BCB)Aₙ]E}ₘ multiblocks used according to the present invention where A is a polymeric polyester unit derived from an aliphatic hydroxy carboxylic acid or a related ester or dimeric ester and is preferably derived from an aliphatic α-hydroxy carboxylic acid or related ester, including a cyclic dimeric ester, such as, for example, lactic acid, lactide, glycolic acid, glycolide, or a related aliphatic hydroxycarboxylic acid or ester (lactone) such as, for example, ε-caprolactone, δ-glutarolactone, δ-valerolactone, γ-butyrolactone and mixtures, thereof, among numerous others as set forth herein. The use of α-hydroxyacids and their corresponding cylic dimeric esters, especially lactide and glycolide in the present invention, is preferred.

In one preferable embodiment, the water-soluble block copolymer of present invention is a tri-block EO-PO-EO copolymer which may be represented by the following general formula:

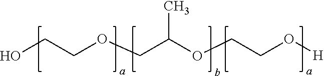

wherein a represents a number from 40 to 160, preferably 50 to 150, more preferably 55 to 145, and b represents a number from 30 to 80, preferably 35 to 70, more preferably 40 to 60.

Preferably, the thermo-sensitive composition according to the invention can comprise greater than or equal to 5% by weight, preferably from 5 to 20% by weight, more preferably from 6 to 18% by weight, most preferably from 7 to 15% by weight, especially from 8 to 13% by weight of component (a), based the total weight of the thermo-sensitive composition.

### Ester/Alkane-Based Emollient (b)

In the context of the present invention, the thermo-sensitive composition comprises at least one ester-based emollient and/or alkane-based emollient as component (b).

The term “emollient” used herein, refers to a material useful for the prevention or relief of dryness, as well as for the protection of the skin.

In the ester-based emollient, the ester is formed from at least one acid and at least one alcohol, and the total number of carbon atoms of the individual acid and the individual alcohol is less than or equal to 42, preferable less than or equal to 28, more preferably less than or equal to 22, and the absolute value of the difference of the number of carbon atoms of the individual acid and the individual alcohol is less than or equal to 15, preferably less than or equal to 10, more preferably less than or equal to 8.

In the ester, the acid moiety can be derived from monocarboxylic acids, dicarboxylic acids, polycarboxylic acids comprising at least 3 carboxyl groups, or carbonic acid.

The monocarboxylic acid can be saturated or unsaturated, or even aromatic, linear, or branched, comprising 5 to 26 carbon atoms, preferably 6 to 22 carbon atoms, more preferably 6 to 18 carbon atoms.

Among the monocarboxylic acids that can be used, the following may be mentioned, alone or mixed:
saturated monocarboxylic acids such as caproic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, caprylic acid, isooctanoic acid, nonanoic acid, isononanoic acid, capric acid, lauric acid, tridecanoic acid, myristic acid, palmitic acid, stearic acid, isostearic acid, arachidic acid, behenic acid, cerotic acid, cocinic acid; and
unsaturated monocarboxylic acids, such as caproleic acid, obtusilic acid, undecylenic acid, dodecylenic acid, linderic acid, myristoleic acid, oleic acid, erucic acid; and aromatic monocarboxylic acids, such as benzoic acid, o-toluic acid, m-toluic acid, p-toluic acid, 4-tert-butyl-benzoic acid.

Preferably, the following may be used: 2-ethylhexanoic acid, caprylic acid, isooctanoic acid, capric acid, lauric acid, myristic acid, isoheptanoic acid, isononanoic acid, nonanoic acid, palmitic acid, stearic acid, oleic acid, erucic acid, cocinic acid, benzoic acid, o-toluic acid, m-toluic acid or combination thereof.

The dicarboxylic acid can be saturated or unsaturated, or even aromatic, linear or branched, comprising 5 to 15 carbon atoms, preferably 6 to 12 carbon atoms, more preferably 6 to 10 carbon atoms.

Preferably, said dicarboxylic acid is aliphatic and comprise 5 to 15 carbon atoms, preferably 6 to 12 carbon atoms, more preferably 6 to 10 carbon atoms.

Among the dicarboxylic acids that can be used, the following may be mentioned, alone or mixed:
decanedioic acid, dodecanedioic acid, suberic acid, oxalic acid, malonic acid, succinic acid, phthalic acid, terephthalic acid, isophthalic acid, pimelic acid, sebacic acid, azelaic acid, glutaric acid, adipic acid, fumaric acid, maleic acid, itaconic acid.

The polycarboxylic acid can be saturated or unsaturated, or even aromatic, linear or branched, comprising at least 3 carboxyl groups, preferably 3 to 4 carboxyl groups, more preferably 3 carboxyl groups.

Said polycarboxylic acid can comprise 5 to 15 carbon atoms, preferably 6 to 12 carbon atoms, more preferably 6 to 10 carbon atoms.

Preferably, said polycarboxylic acid is aliphatic and comprises 5 to 15 carbon atoms, preferably 6 to 12 carbon atoms, more preferably 6 to 10 carbon atoms. Preferably it comprises 3 carboxyl groups.

Among the polycarboxylic acids that can be used, the following may be mentioned, alone or mixed:
tricarboxylic acids, such as trimellitic acid, 1,2,3-benzenetricarboxylic acid, 1,3,5-benzenetricarboxylic acid; and
tetracarboxylic acids such as butanetetracarboxylic acid and pyromellitic acid.

In the ester, the alcohol moiety can be derived from mono-alcohols, diols, or polyols.

The mono-alcohols can be unbranched or branched aliphatic C₁-C₂₅-alcohols, preferably C₂-C₂₂-alcohols, more preferably C₄-C₂₀-alcohols, such as ethanol, propanol, iso-propanol, n-butanol, sec-butanol, iso-butanol, tert-butanol, n-pentanol, iso-pentanol, tert-pentanol, n-hexanol, methyl-2-butanol, 3-methyl-3-pentanol, 2-ethylhexanol, n-heptanol, n-octanol, iso-octanol, 2-ethylhexanol, n-nonanol, iso-nonanol, 2-propylhexanol, n-decanol, iso-decanol, 2-propylheptanol, n-undecanol, n-dodecanol, n-tridecanol, n-tetradecanol, n-pentadecanol or combination thereof.

Preferred mono-alcohols are iso-propanol, n-butanol, n-hexanol, n-octanol, iso-octanol, n-nonanol, iso-nonanol, n-dodecanol, n-tridecanol, n-tetradecanol, n-pentadecanol, cetearyl alcohol, oleic alcohol, octyldodecanol, decyl alcohol or combination thereof.

The diols can be unbranched or branched aliphatic C₂-C₃-alkanediols, more preferably unbranched or branched C₂-C₆-alkanediols, such as 1,2-ethanediol, 1,2-propanediol, 1,3-propanediol, 1,2-butanediol, 1,3-butanediol, 1,4-butanediol, 1,2-pentanediol, 1,3-pentanediol, 2-methyl-1,3-pentanediol, 2,2-dimethyl-1,3-pentanediol, 1,4-pentanediol, 1,5-pentanediol, 1,2-hexanediol, 1,3-hexanediol, 1,4-hexanediol, 1,5-hexanediol, 1,6-hexanediol, or mixtures of these diols. More particularly, the diols used in preparing the polyester plasticizers of the general formula (I) are 1,2-propanediol, 1,3-butanediol, 1,4-butanediol, 2,2-dimethyl-1,3-propanediol, or mixtures thererof.

The polyols can be unbranched or branched aliphatic, comprising 3 to 4 hydroxyl groups.

Said polyol preferably comprises 3 to 15 carbon atoms, preferably 3 to 12, more preferably 4 to 10 carbon atoms.

Among the polyol that can be used, the following may be mentioned, alone or mixed:
triols, such as glycerol, trimethylolethane, trimethylolpropane, 1,2,4-butanetriol, 1,2,6-hexanetriol; and
tetraols, such as pentaerythritol (tetramethylolmethane), erythritol, diglycerol or ditrimethylolpropane.

In one preferable embodiment, the ester is formed from at least one acid selected from the group consisting of caprylic acid, isononanoic acid, capric acid, lauric acid, 2-ethylhexanoic acid, benzoic acid, adipic acid, carbonic acid or combination thereof and at least one alcohol selected from the group consisting of n-butanol, n-hexanol, n-octanol, iso-nonanol, n-dodecanol, n-tridecanol, n-tetradecanol, n-pentadecanol, glycerol, pentaerythritol or combination thereof.

In one particularly preferable embodiment, the ester is selected from the group consisting of dibutyl adipate, hexyl laurate, C₁₂₋₁₅-alkyl benzoate, pentaerythrityl tetraethylhexanoate, triethylhexanoin, dicaprylyl carbonate, isononyl isononanoate, caprylic/capric triglyceride or combination thereof.

As the alkane-based emollient, at least one selected from the group consisting of linear alkanes, paraffin oil and hydrogenated polyisobutene is suitable for the invention.

As examples of alkanes that are suitable for the invention, mention may be made of n-heptane (C₇), n-octane (C), n-nonane (C₉), n-decane (C₁₀), n-undecane (C₁₁), n-dodecane (C₁₂), n-tridecane (C₁₃) and n-tetradecane (C₁₄), n-pentadecane (C₁₅) and mixtures thereof. According to one particular embodiment, the linear alkane is chosen from n-nonane, n-undecane, n-dodecane, n-tridecane, n-tetradecane and n-pentadecane, and mixtures thereof.

According to one preferred embodiment, mention may be made of mixtures of n-undecane (C₁₁) and of n-tridecane (C₁₃) (commercially available as CETIOL® ULTIMATE from BASF).

According to the invention, paraffin oil advantageous according to the invention which may be used is Merkur white oil Pharma 40 from Merkur Vaseline, Shell Ondina 917, Shell Ondina 927, Shell Oil 4222, Shell Ondina 933 from Shell & DEA Oil, Pionier 6301 S, Pionier 2071 (Hansen & Rosenthal).

According to the invention, hydrogenated polyisobutene advantageous according to the invention which may be used is LUVITOL® LITE EM, which is commercially available from BASF.

Preferably, the thermo-sensitive composition according to the invention can comprise from more than 0 to less than or equal to 60% by weight, preferably from 4 to 55% by weight, more preferably from 5 to 50% by weight, most preferably from 10 to 45% by weight, especially from 25 to 45% by weight of component (b), based the total weight of the thermo-sensitive composition.

In one embodiment, the amounts of components (a) and (b) in % by weight satisfy the following equation: (a)+(b)/9≥11.5, preferably 25.0≤(a)+(b)/9≥11.5, more preferably from 15.0≥(a)+(b)/9≥11.5.

In one preferable embodiment, the thermo-sensitive composition according to the invention can comprise greater than or equal to 5% by weight, preferably from 5 to 20% by weight, more preferably from 6 to 18% by weight, most preferably from 7 to 15% by weight, especially from 8 to 13% by weight of component (a), and from more than 0 to less than or equal to 60% by weight, preferably from 4 to 55% by weight, more preferably from 5 to 50% by weight, most preferably from 10 to 45% by weight, especially from 25 to 45% by weight of component (b), based the total weight of the thermo-sensitive composition, and the amounts of components (a) and (b) in % by weight satisfy the following equation: (a)+(b)/9≥11.5, preferably 25.0≥(a)+(b)/9≥11.5, more preferably from 15.0≥(a)+(b)/9≥11.5.

### Emulsifier (c)

In the context of the present invention, the thermo-sensitive composition comprises at least one emulsifier as component (c).

Any conventionally used emulsifier in cosmetic application or personal care application can be used for the present compositions.

Emulsifier may comprise for example:

Carboxylic acids and their salts: alkaline soap of sodium, potassium and ammonium, metallic soap of calcium or magnesium, organic basis soap such as Lauric, palmitic, stearic and oleic acid etc. Alkyl phosphates or phosphoric acid esters, acid phosphate, diethanolamine phosphate, potassium cetyl phosphate. Ethoxylated carboxylic acids or polyethyleneglycol esters, PEG-n acylates. Linear fatty alcohols having from 8 to 22 carbon atoms, branched from 2 to 30 mol of ethylene oxide and/or from 0 to 5 mol propylene oxide with fatty acids having from 12 to 22 carbon atoms and with alkylphenols having from 8 to 15 carbon atoms in the alkyl group. Fatty alcohol polyglycolether such as Beheneth-n (for example Beheneth-25 (Eumulgin® BA 25)), laureth-n, ceteareth-n, steareth-n, oleth-n. Fatty acid polyglycolether such as PEG-n stearate, PEG-n oleate, PEG-n cocoate. Monoglycerides and polyol esters. C₁₂-C₂₂ fatty acid mono- and di-esters of addition products of from 1 to 30 mol of ethylene oxide with polyols. Fatty acid and polyglycerol ester such as monostearate glycerol, diisostearoyl polyglyceryl-3-diisostearates, polyglyceryl-3-diisostearates, triglyceryl diisostearates, polyglyceryl-2-sesquiisostearates, polyglyceryl-2 dipolyhydroxystearate, or polyglyceryl dimerates. Mixtures of compounds from a plurality of those substance classes are also suitable. Fatty acid polyglycolesters such as monostearate diethylene glycol, fatty acid and polyethylene glycol esters, fatty acid and saccharose esters such as sucro esters (for example sucrose polystearate (Emulgade® Sucro)), glycerol and saccharose esters such as sucro glycerides. Sorbitol and sorbitan, sorbitan mono- and di-esters of saturated and unsaturated fatty acids having from 6 to 22 carbon atoms and ethylene oxide addition products. Polysorbate-n series, sorbitan esters such as sesquiisostearate, sorbitan, PEG-(6)-isostearate sorbitan, PEG-(10)-sorbitan laurate, PEG-17-dioleate sorbitan. Glucose derivatives, C₈-C₂₂ alkyl-mono and oligo-glycosides and ethoxylated analogues with glucose being preferred as the sugar component. O/W emulsifiers such as methyl gluceth-20 sesquistearate, sorbitan stearate/sucrose cocoate, methyl glucose sesquistearate, cetearyl alcohol/cetearyl glucoside. W/O emulsifiers such as methyl glucose dioleate/methyl glucose isostearate. Sulfates and sulfonated derivatives, dialkylsulfosuccinates, dioctyl succinate, alkyl lauryl sulfonate, linear sulfonated parafins, sulfonated tetraproplyne sulfonate, sodium lauryl sulfates, amonium and ethanolamine lauryl sulfates, lauyl ether sulfates, sodium laureth sulfates, sulfosuccinates, aceyl isothionates, alkanolamide sulfates, taurines, methyl taurines, imidazole sulfates; polysiloxane/polyalkyl/polyether copolymers and derivatives, dimethicone, copolyols, silicone polyethylene oxide copolymer, silicone glycol copolymer; propoxylated or POE-n ethers (Meroxapols); zwitterionic surfactants that carry at least one quaternary ammonium group and at least one carboxylate and/or sulfonate group in the molecule. Zwitterionic emulsifiers that are especially suitable are betaines, such as N-alkyl-N,N-dimethylammonium glycinates, cocoalkyldimethylammonium glycinate, N-acylaminopropyl-N,N-dimethylammonium glycinates, cocoacylaminopropyldimethylammonium glycinate and 2-alkyl-3-carboxymethyl-3-hydroxyethylimidazolines each having from 8 to 18 carbon atoms in the alkyl or acyl group and also cocoacylaminoethylhydroxyethylcarboxymethylglycinate, N-alkylbetaine, N-alkylaminobetaines. alkylimidazolines, alkylopeptides, lipoaminoacides, self-emulsifying bases and the compounds as described in K. F. DePolo, A short textbook of cosmetology, Chapter 8, Table 8-7, p250-251.

In one embodiment, the emulsifier may comprise for example non-ionic emulsifiers, such as
(1) products of the addition of 2 to 50 mol ethylene oxide and/or 1 to 20 mol propylene oxide onto linear fatty alcohols containing 8 to 40 carbon atoms, onto fatty acids containing 12 to 40 carbon atoms and onto alkylphenols containing 8 to 15 carbon atoms in the alkyl group;
(2) C₁₂₋₁₃ fatty acid monoesters and diesters of products of the addition of 1 to 50 mol ethylene oxide onto glycerol;
(3) sorbitan monoesters and diesters of saturated and unsaturated fatty acids containing 6 to 22 carbon atoms and ethylene oxide adducts thereof;
(4) alkyl mono- and oligoglycosides containing 8 to 22 carbon atoms in the alkyl group and ethoxylated analogs thereof;
(5) products of the addition of 7 to 60 mol ethylene oxide onto castor oil and/or hydrogenated castor oil;
(6) polyol esters and, in particular, polyglycerol esters such as, for example, polyolpoly-12-hydroxystearate, polyglycerol polyricinoleate, polyglyceryl-4-laurate, polyglycerol diisostearate or polyglycerol dimerate. Mixtures of compounds from several of these classes are also suitable;
(7) products of the addition of 2 to 15 mol ethylene oxide onto castor oil and/or hydrogenated castor oil;
(8) partial esters based on linear, branched, unsaturated or saturated C₆₋₂₂ fatty acids, ricinoleic acid and 12-hydroxystearic acid and polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose) or mixed esters, as well as sucrose fatty acid polyester (for example sucrose esterified with C₈-C₂₀, or C₁₂-C₁₈, or C₁₄-C₁₈ fatty acids), for example sucrose polystearate (commercially available as Emulgade® SUCRO from BASF);
(9) polysiloxane/polyalkyl polyether copolymer or corresponding derivatives; and
(10) mixed esters of pentaerythritol, fatty acids, citric acid and fatty alcohol and/or mixed esters of fatty acids containing 6 to 22 carbon atoms, methyl glucose and polyols, preferably glycerol or polyglycerol.

In one embodiment, the emulsifier may comprise for example anionic emulsifiers such as PEG-2 stearate SE, glyceryl stearate SE, propylene glycol stearate. Anionic acid bases such as cetearyl Alcohol and Sodium cetearyl sulfate, cetearyl alcohol and sodium lauryl sulfate, trilaneth-4 phosphate and glycol stearate and PEG-2 stearate, glyceryl stearate and sodium lauryl sulfate, sodium stearoyl glutamate (for example Eumulgin® SG). Cationic acid bases such as cetearyl alcohol and cetrimonium bromide.

In one preferable embodiment, the composition comprises at least one nonionic emulsifier, wherein the at least one nonionic surfactant can be the alkoxylated nonionic surfactant or non-alkoxylated nonionic surfactant. preferably the alkyoxylated nonionic surfactant is C₁₂-C₂₄ fatty acid ethoxylate, more preferably C₁₆-C₂₂ fatty acid ethoxylates, wherein the non-alkoxylated nonionic emulsifier is sucrose fatty acid ester.

In one more preferable embodiment, the composition comprises at least one anionic emulsifier and at least one nonionic emulsifier, wherein the at least one anionic emulsifier is N-acyl amino acid based surfactant. Examples of N-acyl amino acid based surfactants include but are not limited to N-acylated alanine, N-acyl glutamic acid, N-acryl glycine, N-acrylated sarcosine, and their salts. In particular, the amino acid based surfactant can be a C₈-C₁₆ acyl sarcosinate, a C₈-C₁₆ acyl glutamate, a C₈-C₁₆ acyl glycinate, or a combination thereof. The C₈-C₁₆ acyl sarcosinate, C₈-C₁₆ acyl glutamate, and/or C₈-C₁₆ acyl glycinate is an anionic surfactant derived from the amino acid sarcosine, glutamine, or glycine, respectively, and a corresponding C₈-C₁₆ fatty acid. Suitable C₈-C₁₆ acyl sarcosinates include but are not limited to: sodium lauroyl sarcosinate, sodium cocoyl sarcosinate (which is a mixture of sodium C₈-C₁₆ acyl sarcosinates), sodium myristoyl sarcosinate, ammonium lauroyl sarcosinate, ammonium cocoyl sarcosinate, isopropyl lauroyl sarcosinate, potassium cocoyl sarcosinate, potassium lauroyl sarcosinate, and combinations thereof; suitable C₈-C₁₆ glutamates include but are not limited to: sodium lauroyl glutamate, sodium cocoyl glutamate (which is a mixture of sodium C₈-C₁₆ acyl glutamates), sodium myristoyl glutamate, ammonium lauroyl glutamate, ammonium cocoyl glutamate, isopropyl lauroyl glutamate, potassium cocoyl glutamate, potassium lauroyl glutamate, and combinations thereof; suitable C₈-C₁₆ acyl glycinates include but are not limited to: sodium lauroyl glycinate, sodium cocoyl glycinate (which is a mixture of sodium C₈-C₁₆ acyl glycinates), sodium myristoyl glycinate, ammonium lauroyl glycinate, ammonium cocoyl glycinate, isopropyl lauroyl glycinate, potassium cocoyl glycinate, potassium lauroyl glycinate, and combinations thereof. Combinations of one or more C₈-C₁₆ acyl sarcosinates, C₈-C₁₆ acyl glutamates, and/or C₈-C₁₆ acyl glycinates may also be used.

Preferably, the thermo-sensitive composition according to the invention can comprise from 0.1 to 30% by weight, preferably from 0.5 to 25% by weight, more preferably from 1 to 20% by weight, most preferably from 2 to 15% by weight, especially from 3 to 10% by weight of the emulsifier, based the total weight of the thermo-sensitive composition.

In other preferable embodiments, the thermo-sensitive composition according to the invention further comprise the following other ingredients.

### Thickeners

Suitable thickeners for the thermo-sensitive composition according to the invention are polysaccharides, preferably xanthan gum (for example Rheocare® XGN (commercially available from BASF)), guar gum, agar, alginates or tyloses, cellulose derivatives, for example hydroxyalkyl-cellulose, wherein alkyl is a C₁-C₄-alkyl, particularly hydroxyethyl-cellulose, preferably the Natrosol™ trademarks, especially preferably Natrosol™ 250 (CAS-Nr. 9004-62-0) of Herkules Incorporated, carboxymethylcellulose or hydroxycarboxymethylcellulose, starches, preferably the trademark National 465, Purity W or starch B990, and also relatively high molecular weight polyethylene glycol mono- and diesters of fatty acids, fatty alcohols, monoglycerides and fatty acids.

Suitable thickeners are also polyacrylates, crosslinked polyacrylic acids and derivatives thereof, such as Tinovis® CD, and Rheocare® TTA (commercially available from BASF), Carbopol® (commercially available from Lubrizol), Ultrez® (commercially available from Lubrizol), Luvigel® EM (commercially available from BASF), Cosmedia® SP (commercially available from BASF), Tinovis® GTC UP (commercially available from BASF), Capigel®98 (commercially available from Seppic), Synthalene® (commercially available from Sigma), the Aculyn® grades from Rohm and Haas, such as Aculyn® 22 (copolymer of acrylates and methacrylic acid ethoxylates with stearyl radical (20 ethylene oxide (EO) units)) and Aculyn® 28 (copolymer of acrylates and methacrylic acid ethoxylates with behenyl radical (25 EO units)).

Suitable thickeners are furthermore, for example, aerosol grades (hydrophilic silicas), polyacrylamides, polyvinyl alcohol and polyvinylpyrrolidone, ethoxylated fatty acid glycerides, esters of fatty acids with polyols, such as, for example, pentaerythritol or trimethylolpropane, fatty alcohol ethoxylates with a narrowed homolog distribution or alkyl oligoglucosides, and also electrolytes such as sodium chloride and ammonium chloride.

The thickeners may be present in a conventional amount in the art and depending on their types. The person skilled in the art will be able to select the appropriate thickener and amount for the present invention.

In the context of the present invention, the thickeners may be present in an amount of from 0.01 to 3 wt %, preferably from 0.05 to 2.0 wt %, more preferably from 0.2 to 1.0 wt %, based on the total weight of the thermo-sensitive composition.

### Preservatives

The thermo-sensitive composition according to the invention can advantageously comprise one or more preservatives.

Advantageous preservatives within the context of the present invention are, for example, formaldehyde donors (such as, for example, DMDM hydantoin, which is commercially available, for example, under the trade name Glydant® (commercially available from Lonza)), iodopropyl butylcarbamates (for example Glycacil-L®, Glycacil-S® (commercially available from Lonza), Dekaben®LMB (commercially available from Jan Dekker)), parabens (p-hydroxybenzoic acid alkyl esters, such as, for example, methyl, ethyl, propyl and/or butylparaben), dehydroacetic acid (Euxyl® K 702 (commercially available from Schulke & Mayr), phenoxyethanol, ethanol, benzoic acid, or combination thereof. So-called preservation aids, such as, for example, octoxyglycerol, glycine, soya etc., are also advantageously used.

Also advantageous are preservatives or preservation aids customary in cosmetics, such as dibromodicyanobutane (2-bromo-2-bromomethylglutarodinitrile), phenoxyethanol, 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzyl alcohol, salicylic acid and salicylates.

The preservatives may be present in an amount of from 0.01 to 5 wt %, preferably from 0.1 to 2 wt %, more preferably from 0.2 to 1 wt %, based on the total weight of the thermo-sensitive composition.

### Perfume Oils

If appropriate, the thermo-sensitive composition according to the invention can comprise perfume oils. Perfume oils which may be mentioned are, for example, mixtures of natural and synthetic fragrances. Natural fragrances are extracts from flowers (lily, lavender, rose, jasmine, neroli, Ylang-Ylang), stems and leaves (Geranium, patchouli, petit grain), fruits (anis, coriander, caraway, juniper), fruit peels (bergamot, lemon, orange), roots (mace, Angelica, celery, cardamom, Costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (Galbanum, elemi, benzoe, myrrh, olibanum, opoponax). Also suitable are animal raw materials, such as, for example, civet and Castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, 4-tert-butyl cyclohexyl acetate, linalyl acetate, dimethylbenzylcarbinyl acetate, phenylethyl acetate, linalyl benzoate, benzyl formate, ethylmethyl phenylglycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonat, the ketones include, for example, the ionones, cc-isomethylions and methyl cedryl ketone, the alcohols include anethol, citronellol, eugenol, isoeugenol, geraniol, linalool, phenylethyl alcohol and terioneol, and the hydrocarbons include primarily the terpenes and balsams. However, preference is given to using mixtures of different fragrances which together produce a pleasing scent note. Essential oils of lower volatility, which are mostly used as aroma components, are also suitable as perfume oils, for example sage oil, chamomile oil, oil of cloves, melissa oil, mint oil, cinnamon leaf oil, linden blossom oil, juniper berry oil, vetiver oil, olibanum oil, Galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzyl acetone, cyclamenaldehyde, linalool, Boisambrene® Forte, ambroxan, indol, hedione, sandelice, lemon oil, mandarin oil, orange oil, allyl amyl glycolate, cyclovertal, lavandin oil, clary sage oil, β-damascone, Geranium oil bourbon, cyclohexyl salicylate, Vertofix® Coeur, iso E-Super©, Fixolide® NP, evernyl, iraldein gamma, phenylacetic acid, geranyl acetate, benzyl acetate, rose oxide, romillat, irotyl and floramat alone or in mixtures.

The perfume oils may be present in the thermo-sensitive composition in a conventional amount.

### Active Ingredients

It has been found that active ingredients of varying solubility can be homogeneously incorporated into the thermo-sensitive composition according to the invention.

According to the invention, the active ingredients can advantageously be selected from a group of NO synthase inhibitors, particularly if the thermo-sensitive composition according to the invention are to serve for the treatment and prophylaxis of the symptoms of intrinsic and/or extrinsic aging and also for the treatment and prophylaxis of the harmful effects of ultraviolet radiation on the skin. A preferred NO synthase inhibitor is nitroarginine.

Furthermore, the active ingredients are advantageously selected from a group consisting of catechins and bile acid esters of catechins and aqueous or organic extracts from plants or parts of plants which have a content of catechins or bile acid esters of catechins, such as, for example, the leaves of the plant family Theaceae, in particular of the species Camellia sinensis (green tea). Their typical ingredients (for example polyphenols or catechins, caffeine, vitamins, sugars, minerals, amino acids, lipids) are particularly advantageous.

Catechins are a group of compounds which are to be regarded as hydrogenated flavones or anthocyanidins and are derivatives of “catechins” (catechol, 3,3′,4′,5,7-flavanpentaol, 2-(3,4-dihydroxyphenyl)chroman-3,5,7-triol). Epicatechin ((2R,3R)-3,3′,4′,5,7-flavanpentaol) is also an advantageous active ingredient within the context of the present invention.

Also advantageous are plant extracts with a content of catechins, in particular extracts of green tea, such as, for example, extracts from leaves of the plants of the Camellia spec. species, very particularly of the tea varieties Camellia sinenis, C. assamica, C. taliensis and C. inawadiensis and hybrids of these with, for example, Camellia japonica.

Preferred active ingredients are also polyphenols and catechins from a group consisting of (−) catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (often also collectively called “flavones”) are also advantageous active ingredients within the context of the present invention.

Further preferred active ingredients are sericoside, pyridoxol, vitamin K, biotin and aroma substances, bisabolol (bisabolol rac. (commercially available from BASF)), tocopherol (Covi-ox® T-50 C (commercially available from BASF)), retinyl palmitate (Vitamin A-Palmitate Care (commercially available from BASF)), retinol (Retinol 50 C (commercially available from BASF)), Schizandra Chinensis Fruit Extract (Sqisandryl® LS 9905 (commercially available from BASF)).

Furthermore, the active ingredients can also very advantageously be selected from a group of hydrophilic active ingredients, in particular from the following group:
α-hydroxy acids, such as lactic acid or salicylic acid and salts thereof, such as, for example, Na-lactate, Ca-lactate, TEA-lactate, urea, allantoin, serine, sorbitol, milk proteins, panthenol, chitosan, glycerin and glyceryl glucoside (for example Hydagen® Aquaporin (commercially available from BASF)) or combination thereof.

The list of specified active ingredients and active ingredient combinations which can be used in the thermo-sensitive composition according to the invention is not of course intended to be limiting. The active ingredients can be used individually or in any combinations with one another.

The active ingredients may be present in the thermo-sensitive composition according to the invention in a conventional amount in the art depending on their types. The person skilled in the art will be able to select the appropriate active ingredients and amounts for the present invention.

The specified and further active ingredients which can be used in the thermo-sensitive composition according to the invention are given in DE 103 18 526 A1 on pages 12 to 17, to which reference is made at this point in its entirety.

### Additional Benefit Agent

The compositions of the present invention may further comprise one or more benefit agents that can provide a positive and/or beneficial effect to the substrate being cared, e.g. to the skin. The skilled person in the art is able to select according to general knowledge in the art of formulating cosmetic compositions, and the vast literature there-related, appropriate such optional ingredients for application purposes.

In one embodiment, the thermo-sensitive composition according to the invention further comprises one or more benefit agents, such as conditioners, skin conditioners, such as vitamins or their derivatives, such as vitamin B complex, including thiamine, nicotinic acid, biotin, pantothenic acid, choline, riboflavin, vitamin B6, vitamin B12, pyridoxine, inositol, carnitine, vitamins A, C, D, E, K and their derivatives, such as vitamin A palmitate, and pro-vitamins, e.g., panthenol (pro vitamin B5), panthenol triacetate and mixtures thereof; antioxidants; free-radical scavengers; abrasives, natural or synthetic; dyes; hair coloring agents; bleaching agents; hair bleaching agents; UV absorbers, such as benzophenone, bornelone, PABA (Para Amino Benzoic Acid), butyl PABA, cinnamidopropyl trimethyl ammonium chloride, disodium distyrylbiphenyl disulfonate, potassium methoxycinnamate; anti-UV agents, such as butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, octyl dimethyl PABA (padimate O), red petrolatum; antimicrobial agents; antibacterial agents, such as bacitracin, erythromycin, triclosan, neomycin, tetracycline, chlortetracycline, benzethonium chloride, phenol, parachlorometa xylenol (PCMX), triclocarban (TCC), chlorhexidine gluconate (CHG), zinc pyrithione, selenium sulfide; antifungal agents; melanin regulators; tanning accelerators; depigmenting agents, such as retinoids such as retinol, kojic acid and its derivatives such as, for example, kojic dipalmitate, hydroquinone and its derivatives such as arbutin, transexamic acid, vitamins such as niacin, vitamin C and its derivatives, azelaic acid, placertia, licorice, extracts such as chamomile and green tea, where retinol, kojic acid, and hydroquinone are preferred; skin lightening agents such as hydroquinone, catechol and its derivatives, ascorbic acid and its derivatives; skin coloring agents, such as dihydroxyacetone; liporegulators; weight-reduction agents; anti-acne agents; anti-seborrhoeic agents; anti-ageing agents; anti-wrinkle agents; keratolytic agents; anti-inflammatory agents; anti-acne agents, such as tretinoin, isotretinoin, motretinide, adapalene, tazarotene, azelaic acid, retinol, salicylic acid, benzoyl peroxide, resorcinol, antibiotics such as tetracycline and isomers thereof, erythromycin, anti-inflammatory agents such as ibuprofen, naproxen, hetprofen, botanical extracts such as Alnus, Arnica, Artemisia capillaris, Asiasarum root, Calendula, chamomile, nidium, comfrey, fennel, Galla rhois, hawthorn, Houttuynia, Hypericum, Jujube, Kiwi, licorice, Magnolia, olive, peppermint, philodendron, Salvia, Sasa albomarginata, imidazoles such as ketoconazole and elubiol; refreshing agents; cicatrizing agents; vascular-protection agents; agents for the reduction of dandruff (Anti-dandruff agent), seborrheic dermatitis, or psoriasis, such as pyrithione salts, being formed from heavy metals such as zinc, tin, cadmium, magnesium aluminum, sodium and zirconium, like zinc pyrithione, shale oil and derivatives thereof such as sulfonated shale oil, selenium sulfide, sulfur, salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazolenitrite and any possible stereo isomers and derivatives thereof such as anthralin, piroctone olamine (Octopirox), selenium sulfide, ciclopirox olamine, anti-psoriasis agents such as vitamin D analogs, e.g. calcipotriol, calcitriol, and tacaleitrol, vitamin A analogs such as esters of vitamin A, including vitamin A palmitate, retinoids, retinols, and retinoic acid, corticosteroids such as hydrocortisone, clobetasone, butyrate, clobetasol propionate; antiperspirants or deodorants, such as aluminum chlorohydrates, aluminum zirconium chlorohydrates; immunomodulators; nourishing agents; depilating agents, such as calcium thioglycolate, magnesium thioglycolate, potassium thioglycolate, strontium thioglycolate; agents for combating hair loss; reducing agents for permanent-waving; reflectants, such as mica, alumina, calcium silicate, glycol dioleate, glycol distearate, silica, sodium magnesium fluorosilicate; essential oils and fragrances.

Needless to say, the thermo-sensitive composition according to the invention should be cosmetically or dermatologically acceptable, i.e., it should contain a non-toxic physiologically acceptable medium and should be able to be applied to the skin. For the purposes of the invention, the expression “cosmetically acceptable” means a composition of pleasant appearance, odor, feel and/or taste.

The pH of the thermo-sensitive composition according to the invention can be conventionally adjusted to for example from 4.0 to 7.0, preferably from 4.5 to 6.5, more preferably from 6.0 to 6.5.

In a second aspect, the present invention provides a method for preparing the thermo-sensitive composition according to the invention, comprising mixing the components of the thermo-sensitive composition.

In a third aspect, the present invention provides the use of the thermo-sensitive composition according to the invention as a delivery system for cosmetically active ingredients.

It has been found that the thermo-sensitive composition according to the present invention can get the expected thermo-sensitive behavior with less content of water-soluble block copolymers of polyethylene oxide and polypropylene oxide in the composition.

### Examples

Aspects of the present invention are more fully illustrated by the following Examples, which are set forth to illustrate certain aspects of the present invention and are not to be construed as limiting thereto.

### Materials:

The materials shown in Table 1 are used.

TABLE 1

| | |
|---|---|
| Trade Name | INCI Name |

(a) Block copolymer

| | |
|---|---|
| Pluracare ® F 127 NF Prill (BASF) | Poloxamer 407 |
| Pluracare ® F 108 NF Prill (BASF) | Poloxamer 338 |

(b) Ester/Alkane

| | |
|---|---|
| CETIOL ® B (BASF) | Dibutyl Adipate |
| CETIOL ® A (BASF) | Hexyl Laurate |
| CETIOL ® AB (BASF) | C₁₂-₁₅ Alkyl Benzoate |
| Cetiol ® PEEH4 SD (BASF) | Pentaerythrityl Tetraethylhexanoate |
| Myritol ® GTEH-SD (BASF) | Triethylhexanoin |
| CETIOL ® CC (BASF) | Dicaprylyl Carbonate |
| CETIOL ® ININ-GL (BASF) | Isononyl Isononanoate |
| MYRITOL ® 312 (BASF) | Caprylic/Capric Triglyceride |
| CETIOL ® V (BASF) | Decyl Oleate |
| CETIOL ® 868 (BASF) | Ethylhexyl Stearate |
| CETIOL ® J 600 (BASF) | Oleyl Erucate |
| CETIOL ® SN (BASF) | Cetearyl Isononanoate |
| ISOPROPYL PALMITATE (BASF) | Isopropyl Palmitate |
| Eutanol ® GM (BASF) | Octyldodecyl Myristate |
| MYRITOL ® 331 (BASF) | Cocoglycerides |
| CETIOL ® ULTIMATE (BASF) | Undecane (and) Tridecane |
| LUVITOL ® LITE (BASF) | Hydrogenated Polyisobutene |
| 10# WHITE OIL (Sinopec) | Mineral Oil |
| CETIOL ® OE (BASF) | Dicaprylyl Ether |
| CEGESOFT ® PFO (BASF) | Passiflora Incarnata Seed Oil |
| CEGESOFT ® PS 6 (BASF) | Olus Oil |
| EUTANOL ® G (BASF) | Octyldodecanol |
| CETIOL ® PGL (BASF) | Hexyldecanol (and) Hexyldecyl Laurate |

(c) Emulsifier

| | |
|---|---|
| Emulgade ® Sucro (BASF) | Sucrose Polystearate (and) Hydrogenated Polyisobutene |
| Eumulgin ® SG (BASF) | Sodium Stearoyl Glutamate |
| Eumulgin ® BA 25 (BASF) | Beheneth-25 |

Thickener

| | |
|---|---|
| Rheocare ® XGN (BASF) | Xanthan Gum |

Preservative

| | |
|---|---|
| Protectol ® PE (BASF) | Phenoxythanol |

### Preparation of the Thermo-Sensitive Composition According to the Invention:

The thermo-sensitive composition according to the invention are prepared as follows and the weight percentages of the materials are shown in Tables 2 to 5 below:
1. Stirring (a) Block copolymer and water at 75-85° C. until uniform;
2. Under agitation, adding Thickener;
3. Adding (c) Emulsifier into (b) Ester/Alkane or water phase to obtain a homogeneous mixture;
4. Heating (b) Ester/Alkane to 75-85° C.;
5. Under agitation, adding water phase to (b) Ester/Alkane;
6. Homogenizing for several minutes;
7. Cooling to below 50° C. under stirring, adding Preservative and adjusting pH to 6-7.

### Texture Transition Temperature Evaluation Method:

1. Putting the sample into a 60 ml plastic bottle in a 4° C. refrigerator for at least 24 h before the test for equilibrium;
2. Putting the sample into a water-bath;
3. Slowly heating up the water-bath from 4 to 40° C., slowly stirring the sample, recording the temperature when the sample turns into cream (no flow under inversion) from flowing lotion as Transition temperature.

The results are shown in Tables 2 to 5 below.

TABLE 2

| Example | Ex 1 | Ex 2 | Ex 3 | Ex 4 | Ex 5 | Ex 6 | Ex 7 | Ex 8 | Ex 9 | Ex 10 | Ex 11 | Ex 12 | Ex 13 | Ex 14 | Ex 15 | Ex 16 |
|---|---|---|---|---|---|---|---|---|---|---|---|---|---|---|---|---|
| Lotion to cream | 21 | 21.9 | 22 | 27.1 | 24 | 28.5 | 19.7 | 22.9 | 37 | 40 | 38 | 38 | 33.1 | 38 | 35 | 40 |
| T/° C. | | | | | | | | | | | | | | | | |
| Water | To | To | To | To | To | To | To | To | To | To | To | To | To | To | To | To |
| | 100 | 100 | 100 | 100 | 100 | 100 | 100 | 100 | 100 | 100 | 100 | 100 | 100 | 100 | 100 | 100 |
| Pluracare ® F 127 | 8 | 8 | 8 | 8 | 8 | 8 | 9 | 9 | 8 | 8 | 8 | 9 | 10 | 8 | 8 | 9 |
| NF Prill | | | | | | | | | | | | | | | | |
| Emulgade ® | 3 | 3 | 3 | 3 | 3 | 3 | 3 | 3 | | | | | | | | |
| Sucro | | | | | | | | | | | | | | | | |
| Eumulgin ® SG | 0.5 | 0.5 | 0.5 | 0.5 | 0.5 | 0.5 | 0.5 | 0.5 | | | | | | | | |
| Rheocare ® XGN | 0.3 | 0.3 | 0.3 | 0.3 | 0.3 | 0.3 | 0.3 | 0.3 | 0.3 | 0.3 | 0.3 | 0.3 | 0.3 | 0.3 | 0.3 | 0.3 |
| CETIOL ® B | 35 | | | | | | | | 35 | | | | | | | |
| CETIOL ® A | | 35 | | | | | | | | 35 | | | | | | |
| CETIOL ® AB | | | 35 | | | | | | | | 35 | | | | | |
| Cetiol ® PEEH4 | | | | 35 | | | | | | | | 35 | | | | |
| SD | | | | | | | | | | | | | | | | |
| Myritol ® GTEH- | | | | | 35 | | | | | | | | 40 | | | |
| SD | | | | | | | | | | | | | | | | |
| CETIOL ® CC | | | | | | 35 | | | | | | | | 35 | | |
| CETIOL ® ININ- | | | | | | | 40 | | | | | | | | 35 | |
| GL | | | | | | | | | | | | | | | | |
| MYRITOL ® 312 | | | | | | | | 40 | | | | | | | | 40 |
| Eumulgin ® | | | | | | | | | 3.5 | 3.5 | 3.5 | 3.5 | 3.5 | 3.5 | 3.5 | 3.5 |
| BA 25 | | | | | | | | | | | | | | | | |
| Protectol ® PE | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |

Adjusting the pH to be 6-7 with 10% citric acid

TABLE 3

| | Example/Comparative Example | | | | | | | | | | | | | | |
|---|---|---|---|---|---|---|---|---|---|---|---|---|---|---|---|
| | Ex | Ex | Ex | Ex | Ex | Ex | Ex | Ex | Ex | Ex | Comp- | Comp- | Comp- | Comp- | Comp- |
| | 17 | 18 | 19 | 20 | 21 | 22 | 23 | 24 | 25 | 26 | Ex 1 | Ex 2 | Ex 3 | Ex 4 | Ex 5 |
| Lotion to cream T/° C. | 26 | 29 | 27.2 | 29.7 | 20.4 | 34 | 24.4 | 25.6 | 26.8 | 23.6 | No | No | No | No | No |
| Water | To | To | To | To | To | To | To | To | To | To | To | To | To | To | To |
| | 100 | 100 | 100 | 100 | 100 | 100 | 100 | 100 | 100 | 100 | 100 | 100 | 100 | 100 | 100 |
| Pluracare ® F 127 NF | 9 | 9 | 9 | 9 | 9 | 9 | 9 | 9 | 9 | 9 | 9 | 9 | 9 | 9 | 9 |
| Prill | | | | | | | | | | | | | | | |
| Emulgade ® Sucro | 3 | 3 | 3 | 3 | 3 | 3 | 3 | 3 | 3 | 3 | 3 | 3 | 3 | 3 | 3 |
| Eumulgin ® SG | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |
| Rheocare ® XGN | 0.3 | 0.3 | 0.3 | 0.3 | 0.3 | 0.3 | 0.3 | 0.3 | 0.3 | 0.3 | 0.3 | 0.3 | 0.3 | 0.3 | 0.3 |
| CETIOL ® 868 | 40 | | | | | | | | | | | | | | |
| CETIOL ® J 600 | | 40 | | | | | | | | | | | | | |
| CETIOL ® V | | | 40 | | | | | | | | | | | | |
| CETIOL ® SN | | | | 40 | | | | | | | | | | | |
| ISOPROPYL | | | | | 40 | | | | | | | | | | |
| PALMITATE | | | | | | | | | | | | | | | |
| Eutanol ® GM | | | | | | 40 | | | | | | | | | |
| CETIOL ® ULTIMATE | | | | | | | 40 | | | | | | | | |
| LUVITOL ® LITE | | | | | | | | 40 | | | | | | | |
| MYRITOL ® 331 | | | | | | | | | 40 | | | | | | |
| 10# WHITE OIL | | | | | | | | | | 40 | | | | | |
| CETIOL ® OE | | | | | | | | | | | 40 | | | | |
| CEGESOFT ® PFO | | | | | | | | | | | | 40 | | | |
| CEGESOFT ® PS 6 | | | | | | | | | | | | | 40 | | |
| EUTANOL ® G | | | | | | | | | | | | | | 40 | |
| CETIOL ® PGL | | | | | | | | | | | | | | | 40 |
| Protectol ® PE | 0.5 | 0.5 | 0.5 | 0.5 | 0.5 | 0.5 | 0.5 | 0.5 | 0.5 | 0.5 | 0.5 | 0.5 | 0.5 | 0.5 | 0.5 |

Adjust the pH to be 6-7 with 10% citric acid
Comp-Ex: Comparative Example;
Ex: Example

TABLE 4

| | Example/Comparative Example | | | | | | | | | |
|---|---|---|---|---|---|---|---|---|---|---|
| | | | | | | | Comp- | Comp- | Comp- | Comp- |
| | Ex 27 | Ex 28 | Ex 29 | Ex 30 | Ex 31 | Ex 32 | Ex 6 | Ex 7 | Ex 8 | Ex 9 |
| Lotion to cream T/° C. | 21.7 | 25 | 23.6 | 26.2 | 21 | 21 | No | No | No | No |
| Water | To 100 | To 100 | To 100 | To 100 | To 100 | To 100 | To 100 | To 100 | To 100 | To 100 |
| Pluracare ® F 127 NF Prill | 6 | 9 | 12 | 12 | 5 | 8 | 7 | 6 | 4 | 8 |
| Emulgade ® Sucro | 3 | 3 | 3 | 3 | 3 | 3 | 3 | 3 | 3 | 3 |
| Eumulgin ® SG | 0.5 | 0.5 | 0.5 | 0.5 | 0.5 | 0.5 | 0.5 | 0.5 | 0.5 | 0.5 |
| Rheocare ® XGN | 0.3 | 0.3 | 0.3 | 0.3 | 0.3 | 0.3 | 0.3 | 0.3 | 0.3 | 0.3 |
| CETIOL ® B | 55 | 30 | 10 | 5 | 60 | 35 | 30 | 45 | 60 | 30 |
| Protectol ® PE | 0.5 | 0.5 | 0.5 | 0.5 | 0.5 | 0.5 | 0.5 | 0.5 | 0.5 | 0.5 |

Adjust the pH to be 6-7 with 10% citric acid
Comp-Ex: Comparative Example;
Ex: Example

TABLE 5

| Example | Ex 33 | Ex 34 | Ex 35 |
|---|---|---|---|
| Lotion to cream T/° C. | 32.1 | 19.1 | 35 |
| Water | To 100 | To 100 | |
| Pluracare ® F 108 NF Prill | 8 | 10 | 10 |
| Emulgade ® Sucro | 3 | 3 | |
| Eumulgin ® SG | 0.5 | 0.5 | |
| Rheocare ® XGN | 0.3 | 0.3 | 0.3 |
| CETIOL ® B | 35 | 40 | |
| CETIOL ® A | | | 40 |
| Eumulgin ® BA 25 | | | 3.5 |
| Protectol ® PE | 0.5 | 0.5 | 0.5 |

Adjusting the pH to be 6-7 with 10% citric acid
Ex: Example

1. A thermo-sensitive composition comprising (a) a water-soluble block copolymer comprising at least two blocks of polyethylene oxide and at least one block of polypropylene oxide; (b) at least one ester-based emollient and/or alkane-based emollient; and (c) at least one emulsifier, wherein said composition has a texture transition temperature from 5 to 40° C.

2. The thermo-sensitive composition according to claim 1, wherein the block copolymer comprises at least two blocks of polyethylene oxide of a formula, —[CH₂CH₂O]ₐ—, and at least one block of polypropylene oxide of a formula, —[CH₂CH(CH₃)O]_{b}—, where a and b each independently represent a number from 10 to 160.

3. The thermo-sensitive composition according to claim 1, wherein the block copolymer is a tri-block EO-PO-EO copolymer which is represented by a following general formula 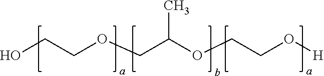
wherein a represents a number from 40 to 160, and b represents a number from 30 to 80.

4. The thermo-sensitive composition according to claim 1, wherein the thermo-sensitive composition comprises greater than or equal to 5% by weight of component (a), based on a total weight of the thermo-sensitive composition.

5. The thermo-sensitive composition according to claim 1, wherein the ester is formed from at least one acid and at least one alcohol, and a total number of carbon atoms of the individual acid and the individual alcohol is less than or equal to 42, and the absolute value of the difference of the number of carbon atoms of the individual acid and the individual alcohol is less than or equal to 15.

6. The thermo-sensitive composition according to claim 5, wherein the acid moiety is derived from monocarboxylic acids, dicarboxylic acids, polycarboxylic acids comprising at least 3 carboxyl groups, or carbonic acid.

7. The thermo-sensitive composition according to claim 6, wherein the monocarboxylic acid is a saturated monocarboxylic acid selected from the group consisting of caproic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, caprylic acid, isooctanoic acid, nonanoic acid, isononanoic acid, capric acid, lauric acid, tridecanoic acid, myristic acid, palmitic acid, stearic acid, isostearic acid, arachidic acid, behenic acid, and cerotic acid; an unsaturated monocarboxylic acid, selected from the group consisting of caproleic acid, obtusilic acid, undecylenic acid, dodecylenic acid, linderic acid, myristoleic acid, oleic acid, and erucic acid; an aromatic monocarboxylic acid selected from the group consisting of benzoic acid, o-toluic acid, m-toluic acid, p-toluic acid, 4-tert-butyl-benzoic acid; and combinations thereof.

8. The thermo-sensitive composition according to claim 6, wherein the dicarboxylic acid is selected from the group consisting of decanedioic acid, dodecanedioic acid, suberic acid, oxalic acid, malonic acid, succinic acid, phthalic acid, terephthalic acid, isophthalic acid, pimelic acid, sebacic acid, azelaic acid, glutaric acid, adipic acid, fumaric acid, maleic acid, itaconic acid, and combinations thereof.

9. The thermo-sensitive composition according to claim 5, wherein the alcohol moiety is derived from mono-alcohols, diols, or polyols.

10. The thermo-sensitive composition according to claim 9, wherein the mono-alcohol is selected from the group consisting of iso-propanol, n-butanol, n-hexanol, n-octanol, iso-octanol, n-nonanol, iso-nonanol, n-dodecanol, n-tridecanol, n-tetradecanol, n-pentadecanol, cetearyl alcohol, oleic alcohol, octyldodecanol, decyl alcohol, and combinations thereof.

11. The thermo-sensitive composition according to claim 9, wherein the diol is an unbranched or branched aliphatic C2-C6-alkanediol selected from the group consisting of 1,2-ethanediol, 1,2-propanediol, 1,3-propanediol, 1,2-butanediol, 1,3-butanediol, 1,4-butanediol, 1,2-pentanediol, 1,3-pentanediol, 2-methyl-1,3-pentanediol, 2,2-dimethyl-1,3-pentanediol, 1,4-pentanediol, 1,5-pentanediol, 1,2-hexanediol, 1,3-hexanediol, 1,4-hexanediol, 1,5-hexanediol, 1,6-hexanediol, and mixtures of these diols.

12. The thermo-sensitive composition according to claim 9, wherein the polyol is selected from the group consisting of glycerol, trimethylolethane, trimethylolpropane, 1,2,4-butanetriol, 1,2,6-hexanetriol, pentaerythritol, erythritol, diglycerol, and ditrimethylolpropane.

13. The thermo-sensitive composition according to any of claim 1, wherein the alkane is at least one selected from the group consisting of linear alkanes, paraffin oil, and hydrogenated polyisobutene.

14. The thermo-sensitive composition according to claim 1, wherein the thermo-sensitive composition comprises from more than 0 to less than or equal to 60% by weight of component (b), based on a total weight of the thermo-sensitive composition.

15. The thermo-sensitive composition according to claim 1, wherein amounts of components (a) and (b) in % by weight satisfy the following equation: (a)+(b)/9≥11.5.

16. The thermo-sensitive composition according to 1, wherein the thermo-sensitive composition comprises greater than or equal to 5% by weight, of component (a), and from more than 0 to less than or equal to 60% by weight, of component (b), based on a total weight of the thermo-sensitive composition, and wherein amounts of components (a) and (b) in % by weight satisfy the following equation: (a)+(b)/9≥11.5, preferably 25.0≥ (a)+(b)/9≥11.5.

17. The thermo-sensitive composition according to claim 1, wherein the emulsifier comprises nonionic emulsifiers and anionic emulsifiers, and/or the thermo-sensitive composition comprises from 0.1 to 30% by weight of the emulsifier, based on a total weight of the thermo-sensitive composition.

18. A method for preparing the thermo-sensitive composition according to claim 1, comprising mixing components of the thermo-sensitive composition.

19. (canceled)

20. A method of delivering a cosmetically active ingredient to skin or hair comprising applying a cosmetic composition comprising the cosmetically active ingredient and a thermos-sensitive composition of claim 1 to the skin or hair.

21. A cosmetic composition comprising the thermo-sensitive composition of claim 1, an active ingredient, and a carrier.
